(19) United States
(12) Patent Application Publication
(10) Pub. No.: US 2023/0166054 A1
(43) Pub. Date: Jun. 1, 2023

(54) Pressure Based Vessel Locating System

(71) Applicant: Bard Access Systems, Inc., Salt Lake City, UT (US)

(72) Inventors: Liam Doctor, Salt Lake City, UT (US); Joshua D. Sherwood, Cottonwood Heights, UT (US)

(21) Appl. No.: 18/072,310

(22) Filed: Nov. 30, 2022

Related U.S. Application Data
(60) Provisional application No. 63/285,048, filed on Dec. 1, 2021.

(51) Int. Cl. A61M 5/42

(52) U.S. Cl. CPC A61M 5/427; A61M 2207/00; A61M 2005/3128

(57) ABSTRACT

A pressure based vessel locating system including a pressure based vessel locator device coupled to a needle or catheter. The pressure based vessel locator device can include a proximal end having an air permeable membrane, and a first chamber and a second chamber. The first chamber can be separated from a second chamber by an interior wall. Each of the first chamber and the second chamber include a proximal opening and a chamber opening having a pressure activated valve disposed therein. The pressure based vessel locator device can further include an air permeable membrane configured to cover proximal openings of the first chamber and the second chamber. Each of the chambers include a window enabling a user to observe the contents of each chamber. A method of identifying a blood vessel includes visually inspecting each chamber for the presence or absence of blood therein.

### PRIORITY

This application claims the benefit of priority to U.S. Provisional Application No. 63/285,048, filed Dec. 1, 2021, which is incorporated by reference in its entirety into this application.

### BACKGROUND

Determining the identity of a blood vessel that has just been accessed can be difficult. Current methods either use an open-ended needle where a clinician must determine through color, texture, spurt distance of blood or use a device showing flashback of blood flow. The open-ended needle is unhygienic while the device showing flashback does not allow the clinician to detect the color or texture of the blood flow. Correctly identifying the blood vessel as an artery or a vein is crucial during placement of medical devices during patient care. It would be beneficial to the clinician and the patient to be able to quickly and correctly identify the blood vessel. Disclosed herein is a pressure-based vessel detector system and method of use that address the foregoing.

### SUMMARY

Disclosed herein is a medical device that, according to some embodiments, includes (i) a third chamber including a fluid port, (ii) a first chamber fluidly coupled with the third chamber via a first valve, and (iii) a second chamber fluidly coupled with the third chamber via a second valve. The first valve is configured to transition from a normally closed state to an opened state in response to a first pressure across the first valve, and the second valve is configured to transition from a normally closed state to an opened state in response to a second pressure across the second valve, where the second pressure is different from the first pressure, and where the fluid port is configured to receive a body fluid from a patient. In some embodiments, the body fluid is blood.

In some embodiments, the fluid port is configured to couple with a vascular access device, and in some embodiments, the fluid port includes a Luer lock connector.

In some embodiments, the first pressure is defined in accordance with a venous pressure of the patient, and in some embodiments the first pressure is between about 4 mmHg and 40 mmHg.

In some embodiments, the second pressure is defined in accordance with an arterial pressure of the patient, and in some embodiments, the second pressure is greater than about 40 mmHg.

In some embodiments, the first valve includes a first septum extending across an opening between the first chamber and the third chamber, and the second valve includes a second septum extending across an opening between the second chamber and the third chamber.

In some embodiments, the first chamber includes a first vent configured to define an atmospheric pressure within the first chamber, and the second chamber includes a second vent configured to define the atmospheric pressure within the second chamber. In some embodiments, the first and second vents include a hydrophobic membrane configured to inhibit passage of a liquid therethrough.

In some embodiments, the first chamber includes a first exterior wall having a first window, and the second chamber includes a second exterior wall having a second window.

In some embodiments, the device further includes a device body, where the device body includes the first exterior wall, the second exterior wall, and an interior wall disposed between the first chamber and the second chamber.

In some embodiments, the first and second exterior walls define a cylindrical circumference of the device body.

In some embodiments, during use, fluid communication between a vein of the patient and the fluid port causes blood to flow into the first chamber, where the blood within the first chamber is visible through the first window.

In some embodiments, during use, fluid communication between an artery of the patient and the fluid port causes blood to flow into the second chamber, where the blood within the second chamber is visible through the second window.

Also disclosed herein is a method of identifying a blood vessel that, according to some embodiments, includes (i) inserting a needle into a target area of a patient, where the needle is coupled with a medical device that includes a first chamber and a second chamber; (ii) visually inspecting the first chamber and a second chamber for the presence of blood therein; and (iii) determining a location of a tip of the needle with respect to a blood vessel as a result of visually inspecting the first chamber and the second chamber.

In some embodiments of the method, visually inspecting the first chamber and the second chamber includes observing an absence of blood within the first chamber and the second chamber, and determining the location of the tip of the needle includes determining that the tip of the needle is disposed outside of the blood vessel.

In some embodiments of the method, visually inspecting the first chamber and a second chamber includes observing a presence of the blood within the first chamber and an absence of the blood within the second chamber, and determining the location of the tip of the needle includes (i) determining that the tip of the needle is disposed within the blood vessel and (ii) determining that the blood vessel is a vein.

In some embodiments of the method, visually inspecting the first chamber and a second chamber includes observing a presence of the blood within the first chamber and the second chamber, and determining the location of the tip of the needle includes (i) determining that the tip of the needle is disposed within the blood vessel and (ii) determining that the blood vessel is an artery.

Also disclosed herein is a method of manufacturing a blood vessel locating system that, according to some embodiments, includes forming a device body of a pressure based vessel locating device, where the device body includes a first chamber having an open proximal end and a first chamber opening at a distal end of the first chamber; (ii) a second chamber having an open proximal end and a second chamber opening at a distal end of the second chamber, where the second chamber is separated from the first chamber by an interior wall; and (iii) a third chamber disposed a distal end of the device body, where the third chamber is in fluid communication with the first chamber opening and the second chamber opening. The method further includes (i) installing a first septum valve across the first chamber opening, where the first septum valve defines a first valve pressure value; (ii) installing a second septum valve across the second chamber opening, where the second septum valve defines a second valve pressure value; (iii) installing an air permeable membrane across the open proximal ends of the first and second chambers; and (iv) attaching a tip member to the device body at the distal end.

In some embodiments of the manufacturing method, the second valve pressure value is at least two times greater than the first valve pressure value.

In some embodiments, the manufacturing method further includes enclosing the pressure based vessel locating device along with one or more needles within a package.

In some embodiments, the manufacturing method further includes In some embodiments, the manufacturing method further includes sterilizing the pressure based vessel locating device along with one or more needles within the package.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a pressure based vessel locating system, in accordance with some embodiments;

FIG. 2A illustrates a perspective view of a pressure based vessel locator device of the system of FIG. 1, in accordance with some embodiments;

FIG. 2B illustrates a cross-sectional side view of the pressure based vessel locator device, in accordance with some embodiments;

FIG. 2C illustrates a cross-sectional exploded view of the pressure based vessel locator device, in accordance with some embodiments;

FIG. 3A illustrates a plan view of the distal end of the pressure based vessel locator device, in accordance with some embodiments;

FIG. 3B illustrates a plan view of the proximal end of the pressure based vessel locator device, in accordance with some embodiments;

FIGS. 4A-4C illustrate a various perspective views of the pressure based vessel locating system depicting an exemplary method of identifying a blood vessel, in accordance with some embodiments;

FIG. 5 illustrates a flow chart of an exemplary method of identifying a blood vessel, in accordance with some embodiments; and

FIG. 6 illustrates a flow chart of an exemplary method of manufacturing the pressure based vessel locating system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a pressure based vessel locator device disclosed herein includes a portion of the pressure based vessel locator device intended to be near a clinician when the pressure based vessel locator device is used on a patient. Likewise, a “proximal length” of, for example, the pressure based vessel locator device includes a length of the pressure based vessel locator device intended to be near the clinician when the pressure based vessel locator device is used on the patient. A “proximal end” of, for example, the pressure based vessel locator device includes an end of the pressure based vessel locator device intended to be near the clinician when the pressure based vessel locator device is used on the patient. The proximal portion, the proximal-end portion, or the proximal length of the pressure based vessel locator device can include the proximal end of the pressure based vessel locator device; however, the proximal portion, the proximal-end portion, or the proximal length of the pressure based vessel locator device need not include the proximal end of the pressure based vessel locator device. That is, unless context suggests otherwise, the proximal portion, the proximal-end portion, or the proximal length of the pressure based vessel locator device is not a terminal portion or terminal length of the pressure based vessel locator device.

With respect to “distal,” a “distal portion” or a “distal-end portion” of, for example, a pressure based vessel locator device disclosed herein includes a portion of the pressure based vessel locator device intended to be near or in a patient when the pressure based vessel locator device is used on the patient. Likewise, a “distal length” of, for example, the pressure based vessel locator device includes a length of the pressure based vessel locator device intended to be near or in the patient when the pressure based vessel locator device is used on the patient. A “distal end” of, for example, the pressure based vessel locator device includes an end of the pressure based vessel locator device intended to be near or in the patient when the pressure based vessel locator device is used on the patient. The distal portion, the distal-end portion, or the distal length of the pressure based vessel locator device can include the distal end of the pressure based vessel locator device; however, the distal portion, the distal-end portion, or the distal length of the pressure based vessel locator device need not include the distal end of the pressure based vessel locator device. That is, unless context suggests otherwise, the distal portion, the distal-end portion, or the distal length of the pressure based vessel locator device is not a terminal portion or terminal length of the pressure based vessel locator device.

Unless defined otherwise, all technical and scientific terms used herein have the same meaning as commonly understood by those of ordinary skill in the art.

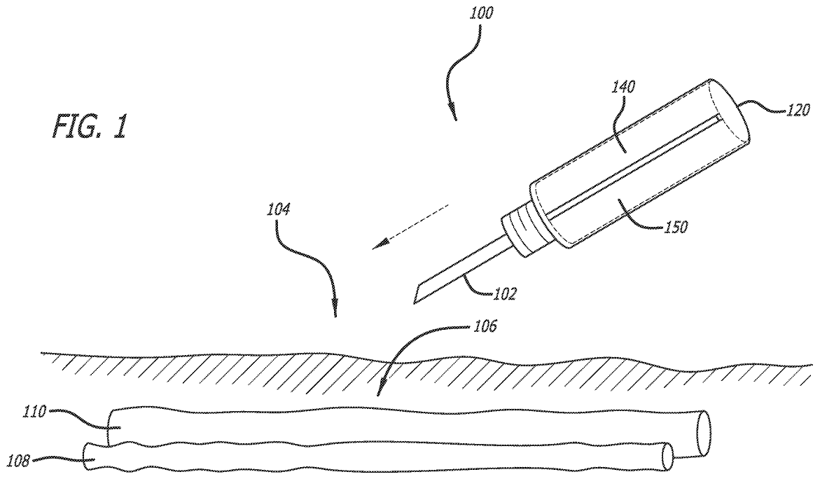

FIG. 1 illustrates a perspective view of a pressure based vessel locating system 100, in accordance with some embodiments. The pressure based vessel locating system (“system”) 100 includes a pressure based vessel locating device (“device”) 120 detachably coupled to a vascular access device, such as a needle 102, for example. The device 120 is generally configured to determine when the tip of the needle 102 is disposed within a blood vessel 106. As the location of the tip of the needle 102 is controlled by the user, the user may thereby determine the location of the blood vessel 106. The device 120 is also generally configured to identify a blood vessel, e.g., determine that the blood vessel is a vein or is an artery. More specifically, the device 120 is configured to identify the blood vessel based on a pressure within the blood vessel. During use, a clinician may access a blood vessel 106, where the blood vessel 106 is one of the vein 110 or the artery 108 via the vascular access device 102, where accessing the blood vessel 106 establishes fluid communication between the blood vessel 106 and device 120.

The device 120 includes a first chamber 140 and a second chamber 150. During use, a user may access the blood vessel 106 via the needle 102. The user may then determine the identity of the blood vessel 106 as an artery 108 or as a vein 110 based upon observing blood within the first chamber 140 and/or the second chamber 150, as will be described in more detail herein.

Although the device 120 is shown and described herein as utilized in determining the location of a vascular device in relation to the vasculature of the patient. The device 120 may also be utilized in determining the location of any tubular device in relation to any anatomical element within a patient where the determination is pressure based.

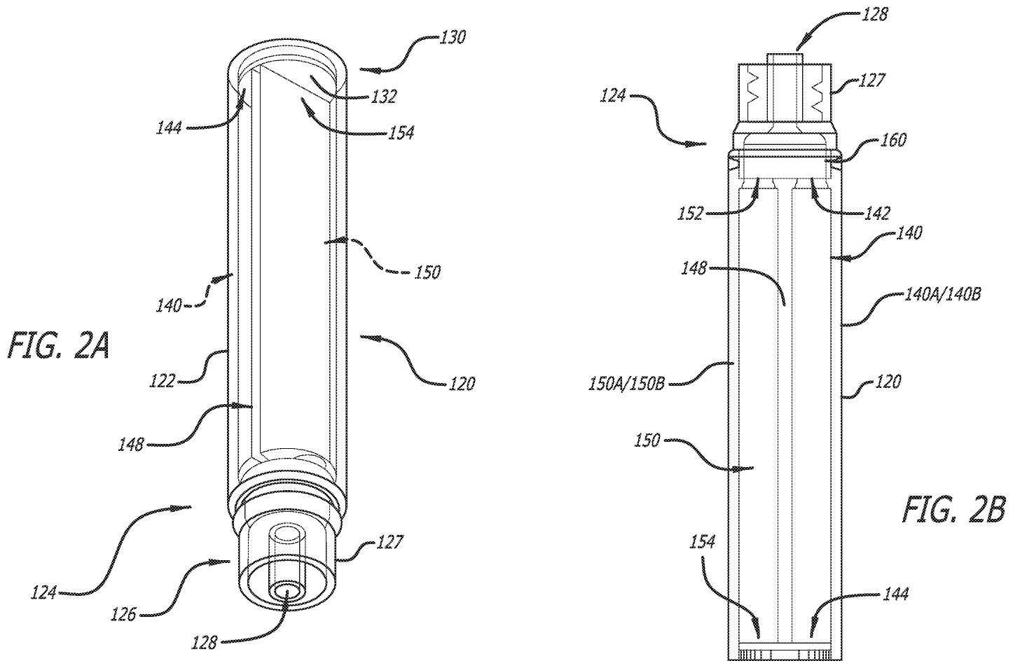

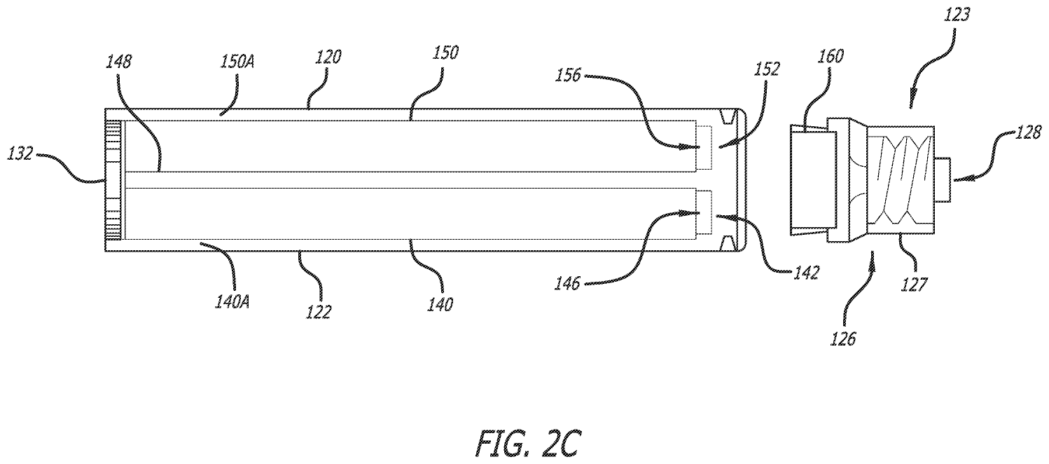

FIG. 2A is a detailed illustration of the device 120. The device 120 may include a body 122 defining the first chamber 140 and the second chamber 150. In some embodiments, the first chamber 140 may be separated from the second chamber 150 by a common interior wall 148. The interior wall 148 may be configured to allow the first chamber 140 to be independent from the second chamber 150. The device 120 may include a distal end 124 having a tip 126 configured to receive thereon the needle 102. The tip 126 includes a tip opening 128. In some embodiments, the tip 126 may include a Luer lock connector 127. The device 120 may include a proximal end 130 wherein the first chamber 140 includes a proximal opening 144 and the second chamber 150 includes a proximal opening 154. In some embodiments, the proximal openings 144/154 may be covered by a membrane 132.

FIG. 2B illustrates a cross-sectional side view of the device 120, in accordance with some embodiments. The device 120 further includes a third chamber 160 in fluid communication with the tip opening 128, where the tip opening 128 defines a fluid port of the third chamber 160. The first chamber 140 and the second chamber 150 are in fluid communication with the third chamber 160. In some embodiments, blood (or other body liquid) may be received through the tip opening 128, enter the third chamber 160, and further enter into the first chamber 140 and/or the second chamber 150. In some embodiments, the distal end 124 of the body 122 may include a first chamber opening 142 and a second chamber opening 152. The first chamber opening 142 defines fluid communication between the third chamber 160 and the first chamber 140, and the second chamber opening 152 defines fluid communication between the third chamber 160 and the second chamber 150.

The body 122 defines a first exterior wall 140A of the first chamber 140 and a second exterior wall 150A of the second chamber 150. The first exterior wall 140A may be formed of a transparent material (or at least a translucent material) such that the first exterior wall 140A includes a window 140B enabling visual observation of contents (e.g., air or blood) of the first chamber 140. Similarly, the second exterior wall 150A may be formed of a transparent material (or at least a translucent material) such that the second exterior wall 150A includes a second window 150B enabling visual observation of contents (e.g., air or blood) of the second chamber 150. The first and second exterior walls 140A, 150A may define a cylindrical circumference of the device body 122.

FIG. 2C illustrates an exploded view of the device 120, in accordance with some embodiments. Shown are (i) the first chamber opening 142 defining fluid communication between the first chamber 140 and third chamber 160 and (ii) the second chamber opening 152 defining fluid communication between the second chamber 150 and third chamber 160. The first chamber opening 142 includes a first valve 146 and the second chamber opening 152 includes a second valve 156. In some embodiments, the first valve 146 and the second valve 156 may each include a septum having a slit. The first valve 146 and the second valve 156 may be normally closed. Each of the first valve 146 and the second valve 156 may be configured to transition from the normally closed configuration to an opened configuration in response to a pressure exerted across the respective valve. The device 120 further includes a tip member 123 which may be coupled to the body 122 during manufacturing, where the tip member 123 includes the Luer lock connector 127, and where the tip member 123 partially defines the third chamber 160.

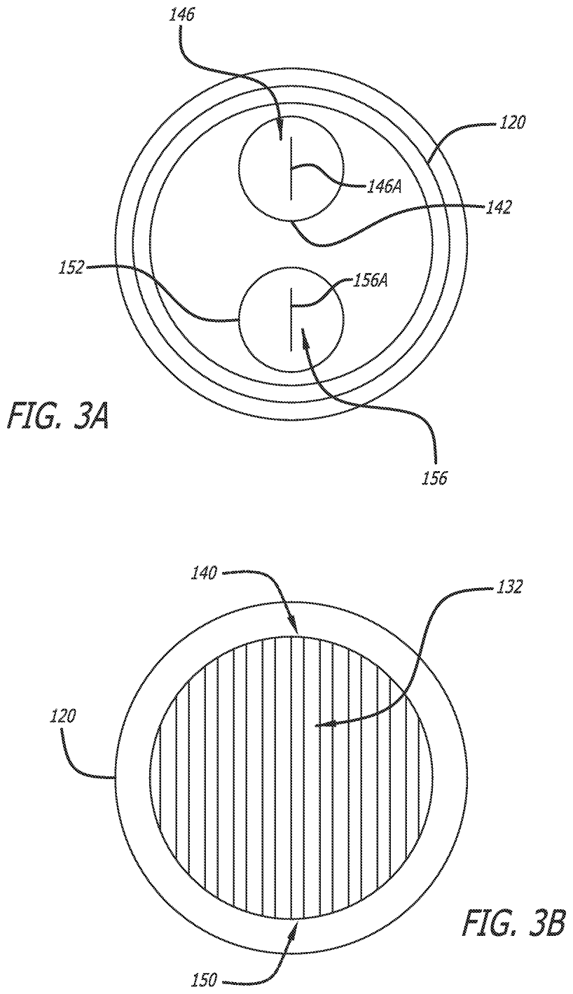

FIG. 3A illustrates an end view of the first chamber opening 142 and the second chamber opening 152, in accordance with some embodiments. Shown are the first valve 146 disposed within the first chamber opening 142 and the second valve 156 disposed within the second chamber opening 152. The first chamber opening 142 and the second chamber opening 152 may be configured in any shape, including a circle, a square, a triangle, a pentagon, or the like. The first valve 146 and the second valve 156 may be constructed of any suitable septum material, such as silicone, rubber, thermoplastic elastomers, or synthetic polymers, for example. The first valve 146 includes a first slit 146A and the second valve 156 includes a second slit 156A.

In some embodiments, the first valve 146 may define a first valve pressure value and the second valve 156 may define a second valve pressure value. The first valve pressure value is the pressure needed to be exerted on the first valve 146 to transition the first valve 146 from the closed configuration to the opened configuration. In some embodiments, the second valve pressure value is the pressure needed to be exerted on the second valve 156 to transition the second valve 156 from the closed configuration to the opened configuration. In some embodiments, the type of material, the thickness of the material, the slit shape, the slit length, or the like of the first valve 146 and the second valve 156 may contribute to the first valve pressure value and the second valve pressure value. In some embodiments, the second valve pressure value may be greater than the first valve pressure value. In some embodiments, the second valve pressure value may be greater than the first valve pressure value by at least a factor of 2.

In some embodiments, the first valve pressure value may be defined in accordance with a venous pressure (i.e., pressure within a vein) such that (i) a pressure exerted on the first valve 146 (i.e., the pressure within the third chamber 160) that is above the venous pressure transitions the first valve 146 from the closed configuration to the opened configuration and (ii) the first valve 146 remains in the closed configuration when the pressure exerted on the first valve 146 is below the venous pressure. Similarly, in some embodiments, the second valve pressure value may be defined in accordance with an arterial pressure (i.e., pressure within an artery) such that (i) a pressure exerted on the second valve 156 (i.e., the pressure within the third chamber 160) that is above the arterial pressure transitions the second valve 156 from the closed configuration to the opened configuration and (ii) the second valve 156 remains in the closed configuration when the pressure exerted on the second valve 156 is below the venous pressure.

In some embodiments, the second valve pressure value may be greater than about 40 mmHg and the first valve pressure value may less than about 40 mmHg or between about 4 mmHg and 40 mmHg. For example, in an instance where the pressure within the third chamber 160 is equal to 10 mmHg, the first valve 146 may transition the from the closed configuration to the opened configuration, while the second valve 156 remains in the closed configuration. Similarly, in an instance where the pressure within the third chamber 160 is equal to 50 mmHg, the first valve 146 and the second valve 156 may transition the from the closed configuration to the opened configuration.

Accordingly, in an instance where the pressure within the third chamber 160 is greater than the first valve pressure value but less than the second valve pressure value, any fluid (e.g., blood) within the third chamber 160 will flow into the first chamber 140 where it is visible through the first window 140B. Similarly, in an instance where the pressure within the third chamber 160 is greater than both the second valve pressure value and the first valve pressure value, any fluid within the third chamber 160 will also flow into the second chamber 150 where it is visible through the second window 150B.

FIG. 3B illustrates a plan view of the membrane 132, in accordance with some embodiments. The membrane 132 is air-permeable, forming an air vent, configured to define an atmospheric pressure within each of the first chamber 140 and the second chamber 150. In some embodiments, the membrane 132 may also be configured to prevent liquid (e.g., blood) from exiting the first chamber 140 or the second chamber 150. In some embodiments, the membrane 132 may be constructed of synthetic polymers including polytetrafluoroethylene or the like. The membrane 132 may be hydrophobic.

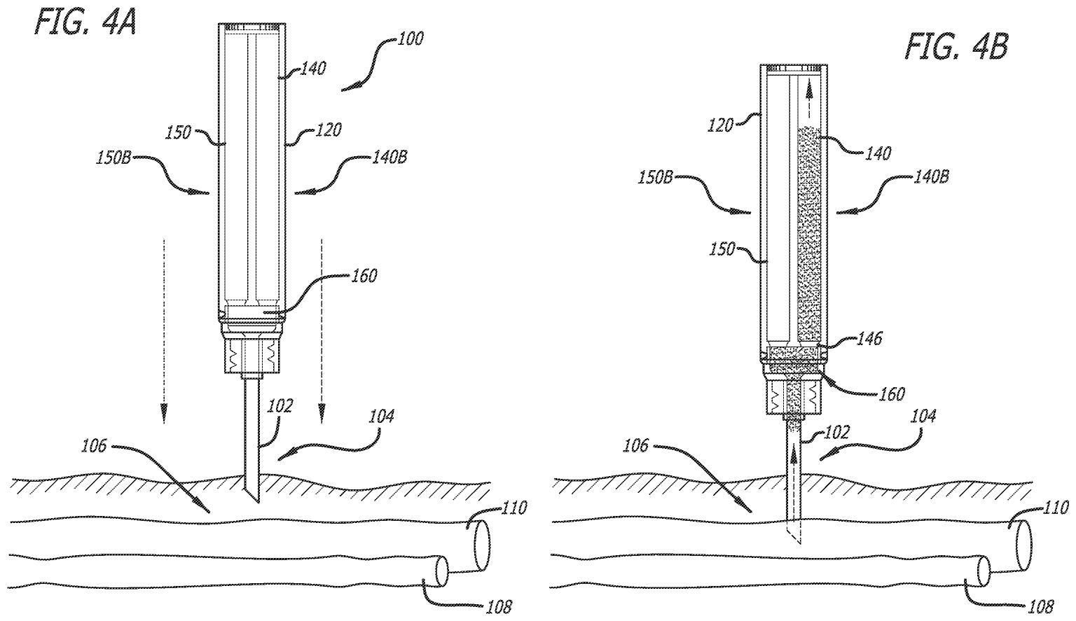

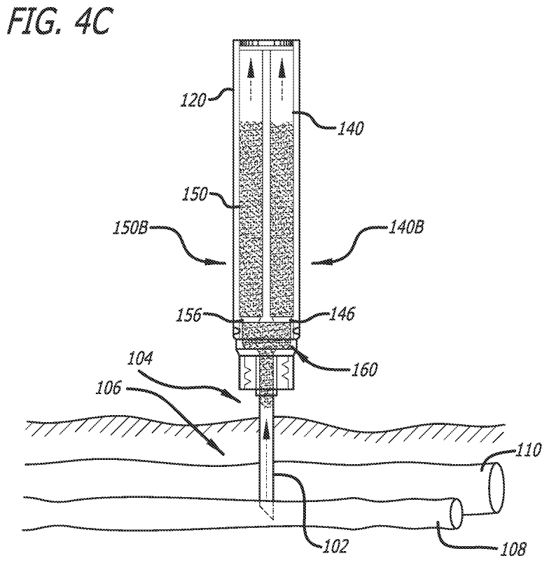

FIGS. 4A-4C illustrate various cross-sectional side views of the device 120 depicting an exemplary method of detecting and/or identifying a blood vessel 106, in accordance with some embodiments. As illustrated in FIG. 4A, the pressure based vessel locating system 100 may be assembled to access a blood vessel 106. In some embodiments, the needle 102 may be coupled to the device 120. The needle 102 is inserted into a target area 104 toward the blood vessel 106 (i.e., the vein 110 or the artery 108). The needle tip is disposed beneath the skin surface but is not disposed within either the vein 110 or the artery 108. Consequently, the third chamber 160 does not contain any blood, nor does either the first chamber 140 or the second chamber 150. As such, the absence of blood is visible through both the first window 140B and the second window 150B. Therefore, a user may determine that the needle 102 is not inserted into the vein 110 or the artery 108.

FIG. 4B illustrates the needle 102 inserted into the vein 110. As such, blood from the vein 110 has traveled along the needle 102 and into the third chamber 160, where the blood disposed within the third chamber 160 defines a venous pressure within the third chamber 160. The venous pressure within the third chamber 160 has transitioned the first valve 146 from the closed configuration to the opened configuration, allowing the blood to pass from the third chamber 160 into the first chamber 140. The pressure within the third chamber 160 is less than the second valve pressure value, and consequently, the second valve 156 remains closed preventing blood from entering the second chamber 150. As such, the presence of blood is visible through the first window 140B and the absence of blood is visible through the second window 150B. Therefore, the user may determine that the needle 102 is inserted into the vein 110.

FIG. 4C illustrates the needle 102 inserted into the artery 108. As such, blood from the artery 108 has traveled along the needle 102 and into the third chamber 160, where the blood disposed within the third chamber 160 defines an arterial pressure within the third chamber 160. The arterial pressure within the third chamber 160 has transitioned the first valve 146 from the closed configuration to the opened configuration, allowing the blood to pass from the third chamber 160 to the first chamber 140. The arterial pressure within the third chamber 160 has also transitioned the second valve 156 from the closed configuration to the opened configuration, allowing the blood to pass from the third chamber 160 into the second chamber 150. As such, the presence of blood is visible through the first window 140B and the second window 150B. Therefore, the user may determine that the needle 102 is inserted into the artery 108.

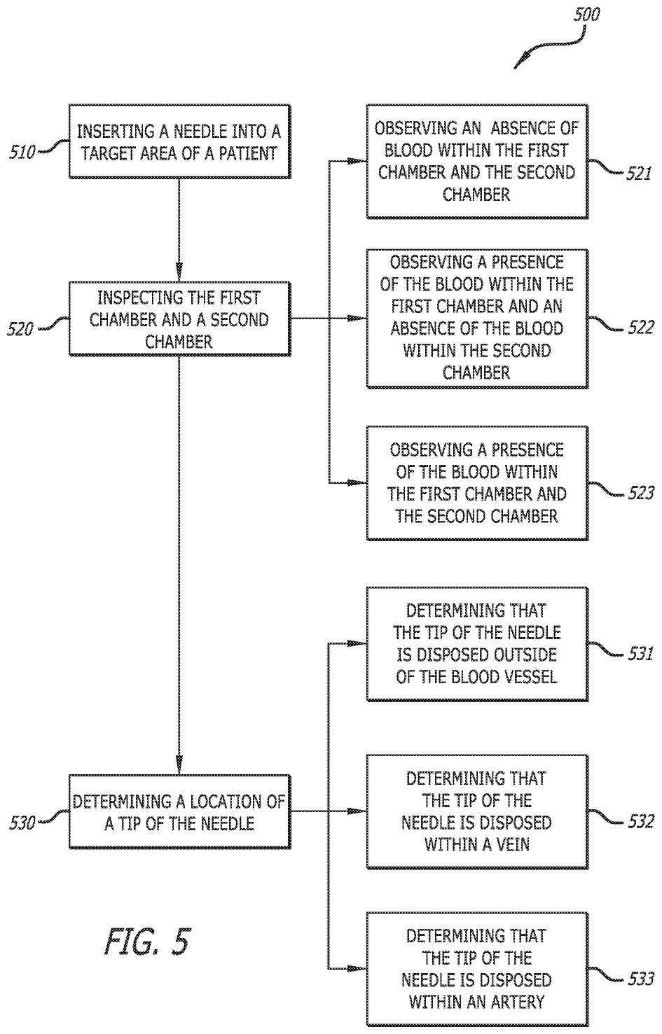

FIG. 5 illustrates a flow chart of an exemplary method 500 of identifying a blood vessel, in accordance with some embodiments. The method 500 may include all or any subset of the following steps, actions, or processes. The method 500 may include inserting a needle into a target area of a patient (block 510), where the needle is coupled with the pressure based vessel locating device that includes the first chamber and the second chamber. The method 500 may further include visually inspecting the first chamber and a second chamber (block 520) and determining a location of a tip of the needle (block 530) with respect to a blood vessel as a result of visually inspecting the first chamber and the second chamber.

In some embodiments of the method 500, visually inspecting the first chamber and the second chamber may include observing an absence of blood within the first chamber and the second chamber (block 521). As result of observing an absence of blood within the first chamber and the second chamber, the method 500 may include determining that the tip of the needle is disposed outside of the blood vessel (block 531).

In some embodiments of the method 500, visually inspecting the first chamber and a second chamber may include observing a presence of the blood within the first chamber and an absence of the blood within the second chamber (block 522). As a result of observing a presence of the blood within the first chamber and an absence of the blood within the second chamber, the method 500 may include (i) determining that the tip of the needle is disposed within the blood vessel and (ii) determining that the blood vessel is a vein (block 532).

In some embodiments of the method 500, visually inspecting the first chamber and a second chamber may include observing a presence of the blood within the first chamber and the second chamber (block 523). As a result of observing a presence of the blood within the first chamber and the second chamber, the method 500 may include (i) determining that the tip of the needle is disposed within the blood vessel and (ii) determining that the blood vessel is an artery (block 533).

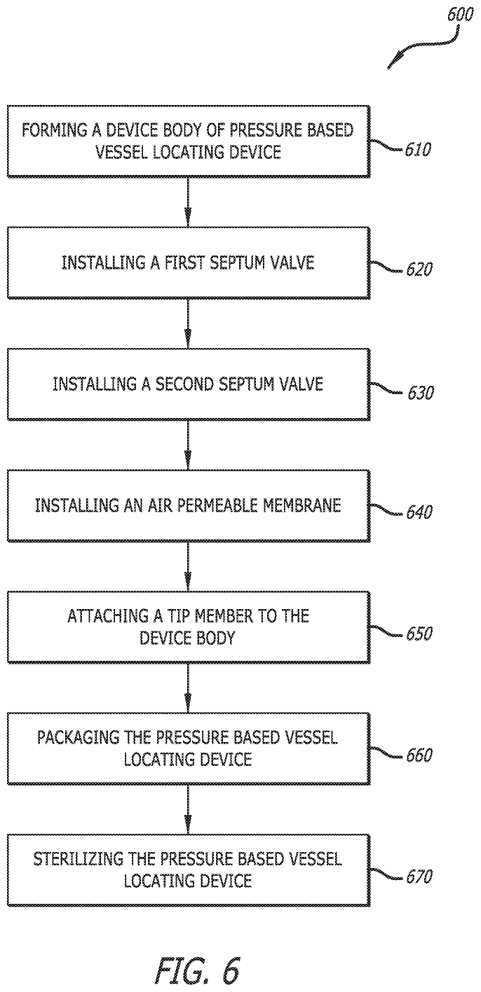

FIG. 6 illustrates a flow chart of an exemplary method 600 of manufacturing the pressure based vessel locating system. The method 600 may include all or any subset of the following steps, actions, or processes. The method 600 may include forming a device body of a pressure based vessel locating device (block 610), where the device body includes a first chamber having an open proximal end and a first chamber opening at a distal end of the first chamber; (ii) a second chamber having an open proximal end and a second chamber opening at a distal end of the second chamber, where the second chamber is separated from the first chamber by an interior wall; and (iii) a third chamber disposed a distal end of the device body, where the third chamber is in fluid communication with the first chamber opening and the second chamber opening. In some embodiments, forming the device body includes injection molding, 3D printing, or extruding the device body. In some embodiments, the device body is formed of a polymer, aluminum, or the like.

The method 600 may further include installing a first septum valve across the first chamber opening (block 620), where the first septum valve defines a first valve pressure value. The method 600 may further include installing a second septum valve across the second chamber opening, where the second septum valve defines a second valve pressure value (block 630). In some embodiments of the manufacturing method, the second valve pressure value is at least two times greater than the first valve pressure value.

The method 600 may further include installing an air permeable membrane (block 640), installing an air permeable membrane includes installing the air permeable membrane across the open proximal ends of the first and second chambers.

The method 600 may further include attaching a tip member to the device body (block 650) at the distal end of the device body.

The method 600 may further include packaging the pressure based vessel locating device (block 660), where packaging the pressure based vessel locating device includes enclosing the pressure based vessel locating device along with one or more needles within a package.

The method 600 may further include sterilizing the pressure based vessel locating device (block 670). In some embodiments, sterilizing the pressure based vessel locating device includes sterilizing the one or more needles along with the pressure based vessel locating device within the package.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device, comprising:
a third chamber including a fluid port;
a first chamber fluidly coupled with the third chamber via a first valve; and
a second chamber fluidly coupled with the third chamber via a second valve,
wherein:
the first valve is configured to transition from a normally closed state to an opened state in response to a first pressure across the first valve,
the second valve is configured to transition from a normally closed state to an opened state in response to a second pressure across the second valve, the second pressure different from the first pressure, and
the fluid port is configured to receive a body fluid from a patient.

2. The device according to claim 1, wherein the body fluid is blood.

3. The device according to claim 1, wherein the fluid port is configured to couple with a vascular access device.

4. The device according to claim 1, wherein the fluid port includes a Luer lock connector.

5. The device according to claim 1, wherein the first pressure is defined in accordance with a venous pressure of the patient.

6. The device according to claim 1, wherein the first pressure is between about 4 mmHg and 40 mmHg.

7. The device according to claim 1, wherein the second pressure is defined in accordance with an arterial pressure of the patient.

8. The device according to claim 1, wherein the second pressure is greater than about 40 mmHg.

9. The device according to claim 1, wherein:
the first valve includes a first septum extending across an opening between the first chamber and the third chamber, and
the second valve includes a second septum extending across an opening between the second chamber and the third chamber.

10. The device according to claim 1, wherein:
the first chamber includes a first vent configured to define an atmospheric pressure within the first chamber, and
the second chamber includes a second vent configured to define the atmospheric pressure within the second chamber.

11. The device according to claim 10, wherein the first and second vents include a hydrophobic membrane configured to inhibit passage of a liquid therethrough.

12. The device according to claim 1, wherein:
the first chamber includes a first exterior wall having a first window, and
the second chamber includes a second exterior wall having a second window.

13. The device according to claim 12, further comprising a device body including:
the first exterior wall;
the second exterior wall; and
an interior wall disposed between the first chamber and the second chamber.

14. The device according to claim 13, wherein the first and second exterior walls define a cylindrical circumference of the device body.

15. The device according to claim 12, wherein during use, fluid communication between a vein of the patient and the fluid port causes blood to flow into the first chamber, the blood within the first chamber visible through the first window.

16. The device according to claim 12, wherein, during use, fluid communication between an artery of the patient and the fluid port causes blood to flow into the second chamber, the blood within the second chamber visible through the second window.

17. A method of identifying a blood vessel, comprising:
inserting a needle into a target area of a patient, the needle coupled with a medical device including a first chamber and a second chamber;
visually inspecting the first chamber and a second chamber for the presence of blood therein; and
determining a location of a tip of the needle with respect to a blood vessel as a result of visually inspecting the first chamber and the second chamber.

18. The method according to claim 17, wherein:
visually inspecting the first chamber and the second chamber includes observing an absence of blood within the first chamber and the second chamber, and
determining the location of the tip of the needle includes determining that the tip of the needle is disposed outside of the blood vessel.

19. The method according to claim 17, wherein:
visually inspecting the first chamber and a second chamber includes observing a presence of the blood within the first chamber and an absence of the blood within the second chamber, and
determining the location of the tip of the needle includes:
determining that the tip of the needle is disposed within the blood vessel,
and
determining that the blood vessel is a vein.

20. The method according to claim 17, wherein:
visually inspecting the first chamber and a second chamber includes observing a presence of the blood within the first chamber and the second chamber,
and
determining the location of the tip of the needle includes:
determining that the tip of the needle is disposed within the blood vessel,
and
determining that the blood vessel is an artery.

21. A method of manufacturing a blood vessel locating system, comprising:
forming a device body of a pressure based vessel locating device, comprising:
a first chamber having an open proximal end and a first chamber opening at a distal end of the first chamber;
a second chamber having an open proximal end and a second chamber opening at a distal end of the second chamber; the second chamber separated from the first chamber via an interior wall; and
a third chamber disposed a distal end of the device body, the third chamber is in fluid communication with the first chamber opening and the second chamber opening;
installing a first septum valve across the first chamber opening, the first septum valve defining a first valve pressure value;
installing a second septum valve across the second chamber opening, the second septum valve defining a second valve pressure value;
installing an air permeable membrane across the open proximal ends of the first and second chambers; and
attaching a tip member to the device body at the distal end.

22. The method according to claim 21, wherein the second valve pressure value is at least two times greater than the first valve pressure value.

23. The method according to claim 21, further comprising enclosing the pressure based vessel locating device along with one or more needles within a package.

24. The method according to claim 23, further comprising sterilizing the pressure based vessel locating device along with one or more needles within the package.
